(19) United States
(12) Patent Application Publication
(10) Pub. No.: US 2025/0375362 A1
(43) Pub. Date: Dec. 11, 2025

(54) DEODORANT COMPOSITIONS

(71) Applicant: THE PROCTER & GAMBLE COMPANY, Cincinnati, OH (US)

(72) Inventors: Edward Dewey SMITH, III, Mason, OH (US); Steven Edward WITT, Morrow, OH (US); David Frederick SWAILE, Cincinnati, OH (US); David Michael EIKE, West Chester, OH (US); Daniel Lawrence CUSTER, West Chester, OH (US)

(21) Appl. No.: 19/228,902

(22) Filed: Jun. 5, 2025

Related U.S. Application Data
(60) Provisional application No. 63/657,481, filed on Jun. 7, 2024.

(51) Int. Cl. A61K 8/34; A61K 8/02; A61K 8/36; A61K 8/49; A61Q 15/00

(52) U.S. Cl. CPC A61K 8/345; A61K 8/0229; A61K 8/361; A61K 8/4926; A61Q 15/00; A61K 2800/30; A61K 2800/5922

(57) ABSTRACT

A deodorant stick composition that contains from about 80% to about 90% of a solvent and a structurant. The solvent includes water and organic solvent that includes a polar organic solvent and a non-polar organic solvent. The polar organic solvent can include 1,3 propanediol and/or glycerin. The ratio of polar organic solvent to non-polar organic solvent is from about 4:5 to about 3:1. The deodorant stick composition can have a hardness from about 80 to about 120 mm*10.

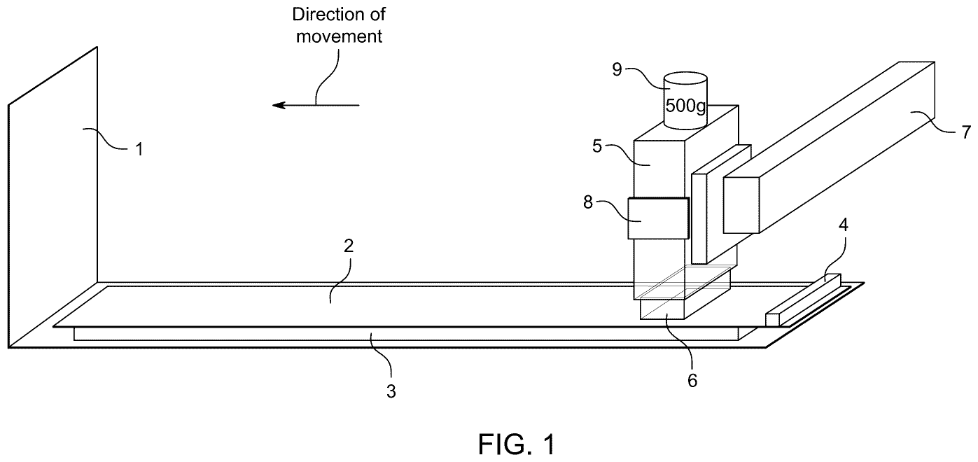

### FIELD OF THE INVENTION

The present disclosure relates to deodorant compositions and methods relating thereto. More particularly, an aluminum-free deodorant stick with a solvent that includes from about 20% to about 50%, by weight of the composition, of a polar organic solvent, such as 1,3-propanediol and/or glycerin.

### BACKGROUND OF THE INVENTION

Many consumers want deodorants that are free of aluminum and are free or have low levels of silicones. These products are specifically formulated to combat body odor by inhibiting the growth of bacteria that cause odor, while also providing a pleasant fragrance to mask body odor throughout the day. One effective method of reducing the growth of microorganisms is to develop deodorant products with a pH level that falls outside the range optimal for microorganism growth when applied to the skin. Furthermore, these products can also be transparent or translucent to provide protection on the skin while being invisible so they can be more aesthetically pleasing to wear.

Some consumers prefer aluminum-free glycol-based deodorant sticks over other forms because they are convenient, quick-drying, transparent or translucent, and are known for being effective at limiting odor and wetness. Furthermore, glycol-based gel sticks can be easier to wash off skin and clothing, as compared to silicone-based sticks that tend to leave behind a waxy residue. However, some individuals have expressed concerns regarding potential irritation caused by deodorant products, especially for those who regularly shave their underarms, as the immediate application of deodorant after shaving is known to exacerbate irritation. Fragrances, high pH levels, and specific solvents are commonly identified as the main culprits of deodorant-related irritation experienced by some users.

Thus, there is a continuing challenge to formulate an aluminum-free, fragrance deodorant stick with low irritation while maintaining sufficient pH control throughout the day to inhibit bacterial growth. Furthermore, the deodorant stick can have both physical and thermal stability, so it does not melt during shipping, handling, or use, and can be processed using standard commercial methods for manufacturing stick deodorants.

### SUMMARY OF THE INVENTION

A deodorant stick composition comprising: (a) from about 80% to about 95% of a solvent comprising: (i) from about 20% to about 50%, by weight of the composition, of a polar organic solvent chosen from 1,3 propanediol, glycerin, or mixtures thereof; (ii) a non-polar organic solvent comprising dipropylene glycol and/or propylene glycol; (iii) water; wherein the ratio of polar organic solvent to non-polar organic solvent is from about 1:3 to about 3:1; (b) greater than 2% and less than 5% of a structurant; and wherein the composition comprises a hardness from about 80 to about 120 mm*10.

A deodorant stick composition comprising: (a) from about 80% to about 95% of a solvent comprising: (i) from about 20% to about 50%, by weight of the composition, of a polar organic solvent chosen from 1,3 propanediol, glycerin, or mixtures thereof; (ii) a non-polar organic solvent comprising dipropylene glycol and/or propylene glycol; (iii) water; wherein the ratio of polar organic solvent to non-polar organic solvent is from about 1:3 to about 3:1; (b) greater than 2% and less than 5% of a structurant; and wherein the composition comprises a hardness from about 80 to about 120 mm*10.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

The FIGURE shows the experimental setup for the Consumption Test Method.

### DETAILED DESCRIPTION OF THE INVENTION

Several well-known companies, such as Procter & Gamble® (known for brands like Secret® and Old Spice®), Unilever® (known for brands like Dove® and Axe®), and Carlyle (known for Every Man Jack®), sell aluminum-free deodorant compositions in a glycol-based stick, often referred to as a gel stick. Some consumers prefer the gel stick product form because in addition to being aluminum-free, it is long-lasting, dries quickly, is moisturizing, is translucent or transparent, rinses off easily, and can be effective at reducing odor throughout the day. The gel stick deodorant is predominantly solvent and also contains a metal salt of a fatty acid as the structurant.

The solvent, which can make up around 80-90% of the total formulation, frequently includes water and/or organic solvents like dipropylene glycol, propylene glycol, and/or glycerin. The solvents can be miscible with each other and are also generally miscible with the fragrance, forming what can be considered a distinct phase of the deodorant stick (i.e., solvent phase or solvent compartment).

The organic solvent has the potential to cause skin irritation in some users. The potential for irritation can be measured in a clinical study such as a 5-day Cumulative Irritation Patch Test (CIPT). Table 2, hereafter, compares Example 1, which does not contain any polar organic solvents, with Examples 2 and 3, which contain 63%, by weight of the organic solvent, of polar organic solvents, with glycerin accounting for 40% of the composition. The skin patch study for Example 1 indicated a relatively high irritation score, while the skin patch study results for Examples 2 and 3 had a low percentage of dropped sites (9%) and a low irritation score of 1.7. Based on these findings, it is believed that using a higher proportion of polar organic solvents in relation to the total organic solvent content has the potential to make the deodorant stick products less irritating to some consumers.

Modifying the solvent in the deodorant stick is challenging because the solvent, when combined with the structurant, directly affects the stick's hardness. When the deodorant stick is too hard, it can be difficult to apply the desired amount of deodorant composition to the underarm. This can result in reduced product performance, fragrance display, and a shorter duration of effectiveness. Additionally, a deodorant stick that is too hard may not glide smoothly over skin during application. Conversely, if the stick is too soft, it wears down quickly, and users may apply an excessive amount of product, resulting in a thick, sticky residue on the skin that may feel greasy and uncomfortable. Striking the right balance in stick hardness is crucial to ensure optimal application, product efficacy, and user comfort.

Example 2 (Table 2, hereafter) includes 5% sodium stearate structurant in combination with the following organic solvents: 40% glycerin, 16.5% dipropylene glycol, and 18.6% propylene glycol. The stick may be considered mild according to the CIPT results. However, it fails to meet consumer acceptability criteria due to its excessive hardness. Example 3 (Table 2, hereafter) has the same organic solvents as Example 2, but the sodium stearate is reduced to 3.5%. As a result, the stick achieves a hardness level that is considered consumer acceptable.

It was found that if the structurant was reduced too much, the composition will not be phase stable because phase separation and weeping can occur. Examples 9, 10, and 11 in Tale 4, hereafter, all have the same organic solvents (17.8% dipropylene glycol, 7.07% propylene glycol, 30% 1-3 propanediol, and 10% glycerin). Examples 9 and 10 include 4% and 3.5% sodium stearate, respectively, and are phase stable and Example 11 includes 3% sodium stearate and weeping was observed on the stick after about 12 hours at ambient conditions.

### Deodorant Stick Composition

The deodorant compositions may have a hardness measured by a penetration value of at most about 120 units, as determined by the test method detailed below. In some embodiments, the hardness may be from about 80 units (mm*10) to about 120 units, from about 85 units to about 115 units as measured by the Hardness Test Method, described hereafter.

The deodorant compositions can have a consumption from about 3.5 mg/cm² to about 5 mg/cm², as determined by the Consumption Test Method, described hereafter. It can be important for a deodorant stick composition to dispense the correct amount of deodorant to ensure effectiveness, efficiency, comfort, and aesthetics. Using the appropriate amount allows for even and adequate application of active ingredients, promoting optimal odor and wetness control without wastage. Moreover, dispensing the correct amount avoids discomfort caused by excessive residue or insufficient coverage, while maintaining a discrete appearance.

Aluminum-free deodorant sticks are most effective if they can change the pH under the arm throughout the day. A pH between 5 and 8 provides optimal conditions for bacterial growth and subsequently underarm malodor. Therefore, it is beneficial for a deodorant to keep the pH of the underarm above at least 8. The deodorant stick composition can have a pH from about 9 to about 10.5, from about 9.5 to about 10, according to the pH Test Method.

### Solvent

The composition can be predominantly solvent. In one example, the solvent can be from about 50% to about 90%, from about 60% to about 90%, from about 70% to about 90%, from about 75% to about 90%, from about 80% to about 90%, or from about 85% to about 90%. The solvents can be miscible. The solvents can include water and organic solvents including polar organic solvents and/or non-polar organic solvents.

The organic solvent can include propylene glycol, dipropylene glycol, tripropylene glycol, low molecular weight polypropylene glycols, 1,3 propanediol, ethylene glycol, diethylene glycol, triethylene glycol, butylene glycol, hexylene glycol, trimethylene glycol, 1, 2 pentanediol, 1,2 hexanediol, butanediol, glycerol, sorbitol, glycerin ethoxylates, and polyethylene glycol including PEG-4 and PEG-8, glycerin, or mixtures thereof.

The deodorant stick compositions can include polar organic solvents and non-polar organic solvents. A solvent is considered polar if it has a Hansen solubility parameter >31 (MPa)\hat{ }0.5. Table 1, below, shows the Hansen Solubility Parameters for common solvents.

TABLE 1

| | Hansen Solubility |
|---|---|
| | Parameter |
| | (MPa)\hat{ }0.5 |
| Water | 47.8 |
| Glycerin | 36.2 |
| 1,3-propanediol | 31.7 |
| Propylene Glycol | 30.2 |
| Dipropylene Glycol | 26.4 |
| Polyethylene Glycol | 19.0 |

The composition can include polar organic solvents and non-polar organic solvents. The composition can include from about 25% to about 70% polar organic solvents, by weight of the organic solvent, from about 30% to about 65%, from about 33% to about 63%, or from about 35% to about 62%. The ratio of polar organic solvent to non-polar organic solvent from about 1:2 to about 3:1, from about 3:5 to about 2:1, or from about 3:5 to about 1.8:1, or from about 4:5 to about 1.7:1.

The composition can include from about 20% to about 50%, from about 22% to about 45%, or from about 25% to about 40%, by weight of the composition, of a polar organic solvent. The polar organic solvent can include 1,3-propanediol, glycerin, xylitol, sucrose, trehalose, isomalt, maltitol, sorbitol, or combinations thereof. The polar organic solvent can include 1,3-propanediol and/or glycerin.

The composition can include from about 12% to about 40%, from about 14% to about 40%, from about 15% to about 33%, or from about 17% to about 30% of 1,3 propanediol.

The composition can include from about 3% to about 15%, from about 4% to about 13%, or from about 5% to about 11% glycerin. The composition can include from about 5% to about 60%, from about 10% to about 58%, from about 25% to about 55%, from about 30% to about 50%, from about 35% to about 45%, or from about 38% to about 42% glycerin.

The composition can include glycerin and 1,3-propanediol at a ratio of 1,3 propanediol to glycerin of from about 1:1 to about 5:1, from about 2:1 to about 4:1, from about 2.5:1 to about 3.5:1, or about 3:1. The composition can include glycerin and 1,3-propanediol at a ratio of 1,3 propanediol to glycerin of from about 1:3 to about 1:1, from about 1:2 to about 1:1, or from about 3:5 to about 1:1.

The composition can include less than 50%, less than 45%, less than 40%, less than 36%, less than 30%, or less than 27%, by weight of the composition, of the non-polar organic solvent. The composition can include from about 10% to about 50%, from about 12% to about 45%, from about 13% to about 40%, from about 15% to about 35%, from about 20% to about 30%, or from about 22% to about 27%, by weight of the composition, non-polar organic solvent. The non-polar organic solvent can include dipropylene glycol and/or propylene glycol.

The composition can include from about 5% to about 35%, from about 10% to about 30%, from about 15% to about 25%, or from about 15% to about 20% of dipropylene glycol. The composition can include from about 20% to about 45%, from about 25% to about 40%, or from about 30% to about 35% dipropylene glycol.

The composition can include from about 3% to about 15%, from about 5% to about 13%, from about 6% to about 12%, or from about 7% to about 10% of propylene glycol.

The solvent can include water. The composition can have from about 5% to about 35% water, from about 10% to about 30%, or from about 15% to about 25% water. The deodorant stick can have a water activity (Aw) from about 0.4 to about 0.85, from about 0.45 to about 0.80, from about 0.5 to about 0.75, or from about 0.55 to about 0.7.

### Fatty Acid Structurants

The deodorant stick compositions also can include a structurant. The structurant can be a fatty acid and/or metal salt of the fatty acid that is added to stick deodorant products and can exist as a distinct phase that forms a hard gel. It can also help reduce underarm sweating and provide antimicrobial benefits.

The fatty acid structurant can form a gel network. When deodorant sticks gelled with sodium stearate are applied to the skin, the gel network is believed to serve as an organic reservoir, remaining on the skin surface, which reduces the ability of irritants such as fragrance molecules to penetrate the stratum corneum, ameliorating the irritation potential of the fragrances.

The composition can include greater than 3% and less than 5%, from about 3.25% to about 4.5%, or from about 3.5% to about 4% of a structurant. The composition can include from about 2% to about 5%, from about 2.25% to about 4.5%, or from about 2.5% to about 4% of a structurant. The structurant can be chosen from stearic acid and salts thereof, soaps and salts thereof, or mixtures thereof. The stearic acid and salts thereof can include sodium stearate, aluminum stearate, magnesium stearate, zinc stearate, 12-hydroxystearate or mixtures thereof. The soaps can include potassium, calcium, magnesium soaps, or mixtures thereof. The structurant can include fatty alcohols, including stearyl alcohol, behenyl alcohol and palmityl alcohol.

Many commercial products include a sodium stearate structurant. Commercial grade sodium stearate is chemically heterogeneous. It is common for its fatty acid mixtures to comprise a mixture of sodium palmitate, sodium stearate, and sodium behenate and still retain the nomenclature “sodium stearate.” In some current products, sodium palmitate is a preferred component of commercial sodium stearate for its ability to provide clarity to the sticks. A commercial example of sodium stearate is designated OP 200 manufactured by Hallstar, Inc. (California, USA) which comprises a mixture of about 2% sodium myristate (C14), 27% sodium palmitate (C16), 35% sodium stearate (C18), and 36% of sodium arachidate (C20)+sodium behenate (C22) and is labeled sodium stearate on commercial deodorant packages.

### Fragrance

Fragrance is a common component of deodorants, and fragrance molecules have varying degrees of polarity, being somewhat or partially soluble in more than one phase. The extent to which any fragrance molecule or mixture is soluble in different locations (phases) within a deodorant stick is called its partitioning behavior and is governed by thermodynamic principles governing solvent-solute interactions. Fragrance molecules can be at least partly soluble in more than one component of a deodorant stick formulation.

The deodorant stick composition can include any fragrance that is cosmetically acceptable. The fragrance may be a liquid at room temperature. A wide variety of chemicals are known as fragrances, including aldehydes, ketones, and esters. More commonly, naturally occurring plant and animal oils and exudates comprising complex mixtures of various chemical components are known for use as fragrances. Generally, the fragrance can be present at a level from about 0.1% to about 30%, from about 0.25% to about 20%, from about 0.5% to about 10%, or from about 1% to about 5%, by weight of the composition.

### Antimicrobials

The deodorant stick may include one or more antimicrobial compositions. For example, antimicrobials may include, without being limited to, piroctone olamine, octenidine HCl, hexamidine, magnesium carbonate, zinc carbonate, thymol, magnesium hydroxide, dead sea salt, magnesium hydroxide and magnesium carbonate hydroxide, calcium carbonate, polyvinyl formate, salycilic acid, niacinamide, phenoxyethanol, eugenol, linolenic acid, dimethyl succinate, citral, triethyl citrate, sepiwhite, baking soda, partially carbonated magnesium hydroxide, magnesium carbonate hydroxide, cinnamon essential oil, cinnamon bark essential oil, cinnamic aldehyde, and combinations thereof.

### Chelator

The deodorant compositions may include a chelator. Specific and/or additional chelators in the present invention may include, but are not limited to, diethylenetriaminepentaacetic acid (DTPA), diethylenetriaminepentakis (methylenephosphonic acid) (DTPMP), desferrioxamine, their salts and combinations thereof, EDTA including disodium EDTA and tetrasodium EDTA, DPTA, EDDS, enterobactin, desferrioxamine, HBED, and combinations thereof. The amount of chelant, by weight of composition, may be from about 0.05% to about 4%.

### Solubilizer

The composition can contain a solubilizer. A suitable solubilizer can be, for example, a surfactant, such as a no-foaming or low-foaming surfactant. Suitable surfactants are nonionic surfactants, cationic surfactants, amphoteric surfactants, zwitterionic surfactants, and mixtures thereof.

Suitable solubilizers include, for example, hydrogenated castor oil, polyoxyethylene 2 stearyl ether, polyoxyethylene 20 stearyl ether, and combinations thereof. One suitable hydrogenated castor oil that may be used in the present composition is polyoxyethylene hydrogenated castor oil.

When the solubilizing agent is present, it is typically present at a level of from about 0.01% to about 5%, from about 0.01% to about 3%, from about 0.05% to about 1%, from about 0.01% to about 0.05%, by weight of the composition.

### Preservatives

The composition can include a preservative. The preservative is included in an amount sufficient to prevent spoilage or prevent growth of inadvertently added microorganisms for a specific period of time, but not sufficient enough to contribute to the odor neutralizing performance of the composition. In other words, the preservative is not being used as the antimicrobial compound to kill microorganisms on the surface onto which the composition is deposited in order to eliminate odors produced by microorganisms. Instead, it is being used to prevent spoilage of the composition in order to increase shelf-life.

The preservative can be any organic preservative material which will not cause damage to fabric appearance, e.g., discoloration, coloration, bleaching. Suitable water-soluble preservatives include organic sulfur compounds, halogenated compounds, cyclic organic nitrogen compounds, low molecular weight aldehydes, parabens, propane diol materials, isothiazolinones, quaternary compounds, benzoates, low molecular weight alcohols, dehydroacetic acid, phenyl and phenoxy compounds, or mixtures thereof.

Non-limiting examples of commercially available water-soluble preservatives include a mixture of about 77% 5-chloro-2-methyl-4-isothiazolin-3-one and about 23% 2-methyl-4-isothiazolin-3-one, a broad spectrum preservative available as a 1.5% aqueous solution under the trade name Kathon® CG by Rohm and Haas Co.; 5-bromo-5-nitro-1,3-dioxane, available under the tradename Bronidox L® from Henkel; 2-bromo-2-nitropropane-1,3-diol, available under the trade name Bronopol® from Inolex; 1,1′-hexamethylene bis(5-(p-chlorophenyl)biguanide), commonly known as chlorhexidine, and its salts, e.g., with acetic and digluconic acids; a 95:5 mixture of 1,3-bis(hydroxymethyl)-5,5-dimethyl-2,4-imidazolidinedione and 3-butyl-2-iodopropynyl carbamate, available under the trade name name Glydant Plus® from Lonza; N-[1,3-bis(hydroxymethyl)2,5-dioxo-4-imidazolidinyl]-N,N′-bis(hydroxy-methyl) urea, commonly known as diazolidinyl urea, available under the trade name Germall® II from Sutton Laboratories, Inc.; N,N″-methylenebis{N′-[1-(hydroxymethyl)-2,5-dioxo-4-imidazolidinyl]urea}, commonly known as imidazolidinyl urea, available, e.g., under the trade name Abiol® from 3V-Sigma, Unicide U-13® from Induchem, Germall 115® from Sutton Laboratories, Inc.; polymethoxy bicyclic oxazolidine, available under the trade name Nuosept® C from Hüls America; formaldehyde; glutaraldehyde; polyaminopropyl biguanide, available under the trade name Cosmocil CQ® from ICI Americas, Inc., or under the trade name Mikrokill® from Brooks, Inc; dehydroacetic acid; and benzsiothiazolinone available under the trade name Koralone™ B-119 from Rohm and Hass Corporation.

Suitable levels of preservative can range from about 0.0001% to about 0.5%, from about 0.0002% to about 0.2%, from about 0.0003% to about 0.1%, by weight of the composition.

The composition can also be preservative free.

### Methods of Use

The deodorant compositions may be topically applied to the axilla or other area of the skin in any known or otherwise effective method for controlling malodor associated with perspiration. These methods comprise applying to the axilla or other area of the human skin an effective amount of the deodorant composition, typically about 3.5 to about 5 mg/cm², and more typically about 4 mg/cm². The deodorant stick composition is generally a leave-on composition that can provide lasting odor protection and freshness. The composition can be substantially free of or free of aluminum salts, parabens, dyes, and/or talc. The deodorant stick can be applied without white marks, in other words, it can go on clear and stay clear.

The combination of solvents, particularly where the solvent contains at least 40% polar organic solvent, by weight of the organic solvent, can provide wetness protection. Wetness protection can refer to the product's ability to help reduce or prevent excessive sweating and the resulting wetness under the arms.

### Definitions

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 21° C. and at ambient conditions, where “ambient conditions” means conditions under about 1 atmosphere of pressure and at about 50% relative humidity. All weights as they pertain to listed ingredients are based on the active level and do not include carriers or by-products that may be included in commercially available materials, unless otherwise specified. All numeric ranges are inclusive of narrower ranges; delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

Except as otherwise noted, the articles “a”, “an”, and “the” mean “one or more”.

Herein, “effective” means an amount of a subject active high enough to provide a significant positive modification of the condition to be treated. An effective amount of the subject active will vary with the particular condition being treated, the severity of the condition, the duration of the treatment, the nature of concurrent treatment, and like factors.

“Substantially free of” refers to about 2% or less, about 1% or less, about 0.5% or less, or about 0.1% or less of a stated ingredient. “Free of” refers to no detectable amount of the stated ingredient or thing.

### Test Methods

### Consumption Test

The Consumption Test measures the amount of deodorant stick product dispensed to a surface using a controlled application procedure. The FIGURE shows the setup for the test method. A Texture Analyzer model TA-xT2i (Stable Micro Systems, USA) with a 50 kg load cell is used for the test. An L-shaped bracket 1 having 40 cm and 20 cm lengths and a depth of 10 cm is bolted to the base of the instrument with the longest bracket dimension parallel to the movement of the instrument. An adjustable product holding bracket 8 is attached to the moving crosshead, the bracket affixes the product 5, which includes the package/dispenser and the deodorant stick, in a vertical, inverted position without gripping the product, i.e., the product is held essentially vertically but has room to easily slide in and out of the bracket and allow all the normal force from the product and added weight to be applied to the application surface. The Texture Analyzer is laid on its side so a product can be held in instrument is adjusted so the long dimension of L-shaped bracket 1 is horizontal and level. The movable head 7 is positioned near the top of L-shaped bracket 1 1. A ½ inch thick rectangular open cell foam piece 3 measuring 24 cm long and 9 cm wide (e.g. soft polyurethane, basis weight about 190 gsm) is placed on the L-bracket 1 and covered with a substrate 2, which is an embossed paper towel having a basis weight of about 60 gsm and is sufficiently strong to survive the test without tearing (e.g., Bounty® Select-A-Size®, half sheet, The Procter & Gamble Company, USA). The paper towel substrate 2 is weighed prior to using it. The paper towel substrate 2 is clipped firmly to the L-bracket 1 to stabilize it under tension of the test, with the foam 3 underneath it, using clamp 4. The product 5 is prepared by elevating it by the package, cutting the deodorant stick 6 using a thin wire dragged firmly across the package rim, wiping loose product debris from the surface due to the cut, and then elevating the deodorant stick 6 by about 1 cm above the package rim, inverting it and placing it into the product holding bracket 8 while maintaining a taught substrate 2 surface that is free of wrinkles. A 500 gram weight 9 is affixed to the package bottom surface (now the top since inverted) using tape or other adhesive, if necessary.

The Texture Analyzer is programmed in compression mode to drag the product 5 for 150 mm across the surface at a speed of 40 mm/second. The product 5 and substrate 2 are dissembled and the substrate 2 is weighed again to determine how much deodorant stick was deposited on the substrate. The increase in weight is determined. The contact area of dispensing is the 150 mm travel length multiplied by the width of the product, plus the area of the product. The area of the product is estimated by measuring the top surface, for example if it is an elliptical shape it is estimated from the two elliptical axes and the equation for the surface area of an ellipse. The amount of stick product dispensed in grams, divided by the contact area in square centimeters, is the consumption for the first test run in mg/cm². The test is repeated 5 times with the product facing the same direction each time. The mean consumption for the 5 test runs is expressed as the deodorant stick product consumption.

### Hardness-Penetration Measurement for Antiperspirant and Deodorant Finished Products

The penetration test is a physical test method that provides a measure of the firmness of waxy solids and extremely thick creams and pastes with penetration values not greater than 250 when using a needle for D1321. The method is based on the American Society for Testing and Materials Methods D-5, D1321 and D217 and DIN 51 579 and is suitable for all solid antiperspirant and deodorant products.

A needle or polished cone of precisely specified dimensions and weight is mounted on the bottom of a vertical rod in the test apparatus. The sample is prepared as specified in the method and positioned under the rod. The apparatus is adjusted so that the point of the needle or cone is just touching the top surface of the sample. Consistent positioning of the rod is critical to the measured penetration value. The rod is then released and allowed to travel downward, driven only by the weight of the needle (or cone) and the rod. Penetration is the tenths of a millimeter travelled following release.

| APPARATUS | SUGGESTED TYPE (OR EQUIVALENT) |
|---|---|
| Penetrometer with Timer | Penetrometer Suitable for ASTM D-5 and D- |
| (see attached drawings | 1321 methods; Examples: Precision or |
| Attachment 1 and | Humboldt Universal Penetrometer (Humboldt |
| Attachment 2) | Manufacturing, Schiller Park, IL USA) or |
| | Penetrometer Model PNR10 or PNR12 |
| | (Petrolab USA or PetroTest GmbH). |
| Penetration Needles | ANTIPERSPIRANT or DEODORANT |
| | SOLIDS can use: |
| | Needles as specified for ASTM Method |
| | D-5, NIST Certified, Fisher Scientific |
| | #01-512. |
| | Needles as specified for ASTM Method |
| | D 1321 /DIN 51 579, Officially certified, |
| | Taper-Tipped needle, No. H-1310, |
| | Humboldt Mfg. |

General Instructions—All Penetrometers-Keep the instrument and needles/probes clean at all times, free from dust and grime. When not in use, store needles in a suitable container to avoid damage. Periodic calibration should confirm:

Electronic Timer is correctly set. Verify against an independent stopwatch if unsure.

Shaft falls without visible signs of frictional resistance.

Ensure the total weight of the shaft and needle is 50±0.2 grams when the shaft is in free fall. Note: for modern, automated or digital systems this may be performed automatically and confirmed through annual calibration.

### At Time of Use Confirm:

Electronic Timer is correctly set to 5.0 seconds.

The appropriate needle is installed and is clean, straight and without obvious defects (visual inspection)

The penetrometer is level and the shaft is clean, straight and falls freely (visual inspection)

Once level, avoid shifting the position of the unit to maintain level.

### Sample Preparation and Measurement

1. On a deodorant or antiperspirant stick that has cooled ambiently to a temperature between 22° C. and 26° C. for at least 24 hours, slice off top ½ inch of product to achieve a flat surface with a wire cutter drawn across the upper lip of the canister.

2. For the first sample to be tested, lubricate the needle by gently wiping with a lint-free tissue coated with a small amount of the product to be tested. This small amount is typically taken from the shaved top.

3. Place the canister in the appropriate location for the measurement. Locate the sample so the needle will penetrate the product 9-11 mm from the inside of the canister wall on the long axis.

4. Using the coarse and fine adjustments, align the height of the penetrometer mechanism head so that the point of the penetrating needle is just touching the surface of the sample.

A weak light at the side of the penetrometer which casts a shadow of the needle on the surface of the sample may be helpful in determining this contact. When a light area on the sample cannot be seen at the end of the tip of the needle's shadow, the needle height over the sample is correctly adjusted. The light should not be strong enough to heat or melt the sample surface. The needle should be just close enough to scratch the sample surface.

5. Perform the penetration measurement at this location by releasing the needle. Record the result.

6. Repeat Steps 2 through 4 at the other test point, i.e., at the other point 9-11 mm inside of the canister wall on the long axis.

To report results, units for penetration are tenths of a millimeter ( 1/10 mm=100 microns). For example, a result of 80 units is 80 mm*10 or 8 mm. Report the average results of at least 4 total measurements from 2 different sticks. Report the average result of the measurements to the nearest tenth of a millimeter.

### pH Test Method

### Materials and Apparatus

PH Meter (calibrated)
Balance (3 place) for measuring finished product & deionized (DI) water
Water bath with ice (to lower samples to 26° C.±1.0° C.) range for measurement
Cutter or razor for slicing finished product in a thin film to maximize surface area
Wide-mouth jar with lid for samples, 40-50 mL range
36° C. convection oven
DI water

### Sample Preparation

First, slice deodorant stick across top of package to expose fresh product for sampling. Then, very thinly slice (<1 mm, 0.40 g±0.1 g) a shaving of finished product into jar. Calculate water necessary for 1% solution in DI water and add. Close the lid. Do not mix, stir, or shake beyond what is necessary for handling & movement of vessel around lab.

Next, place solution (DI water and sample) in the jar in 36° C. convection oven for 1 hour. Remove from oven.

Before pH measurement, use ice water bath to adjust temperature of solution between 26° C.±1.0° C. Remove lid and use probe to check temp as needed.

### pH Measurement

Place the electrode in the 1% solution, gently swirling solution by hand while waiting for reading to stabilize. Record pH once stable (ensuring temperature requirements are met).

### EXAMPLES

The data and examples provided herein help illustrate the deodorant stick compositions described herein. The exemplified compositions are given solely for the purpose of illustration and are not to be construed as limitations of the present disclosure, as many variations thereof are possible without departing from the spirit and scope of the disclosure. All parts, percentages, and ratios herein are by weight unless otherwise specified.

The deodorant stick compositions described below were made by heating all ingredients to above their melt point and using typical mixing procedures known to those of ordinary skill in the art. All ingredients, excluding fragrance, were heated to 85° C. When all materials were homogeneous the formulation was held at the elevated temperature for a minimum of 10 minutes. The temperature was reduced, at which point the fragrance was added. The formulation was poured into a canister suitable for a gel or stick underarm product and then allowed to cool.

Tables 2, 3, and 4 include the calculated % polar organic solvents of the total organic solvents. Solvents were considered polar if they have a Hansen solubility parameter >31 (MPa)\hat{ }0.5. Water was excluded from the calculation since it is not an organic solvent. In the examples below, the polar organic solvents included 1,3-propanediol and glycerin and the non-polar organic solvents included dipropylene glycol and propylene glycol.

Tables 2, 3, and 4 include the results of a 5-day Cumulative Irritation Patch Test (CIPT) conducted to assess the potential irritancy of the stick deodorant products. In the CPIT, 200 mg of deodorant product was applied to a patch which is adhesively attached to the skin on the back of the arm, a new patch is applied daily for 5 days. Dermatological assessments were performed, which included irritation scoring on a scale ranging from 1 to 4. A score of 4 indicated relatively high irritation and a score of 1 indicated no or low irritation. Patches were reapplied after evaluations, but testing is discontinued at any site when irritation reaches a prescribed level. The frequency of discontinued sites for a treatment in a study is therefore also a measurement of irritation.

Tables 2, 3, and 4 include hardness data, as determined by the Hardness Test Method, described herein. The example was considered consumer acceptable if the hardness was from 80 to 115 mm*10, and the example may be consumer preferred if the hardness is from 85 to 100 mm*10.

Tables 2, 3, and 4 include consumption data, as determined by the Consumption Test Method described herein. The deodorant stick has acceptable consumption if it deposited 3-4.5 mg/cm².

Tables 2, 3, and 4 includes physical stability. Compositions were evaluated for physical stability after approximately 12 hours at ambient conditions. A deodorant stick was considered phase stable if by visual detection there is no phase separation or weeping, which occurs when there are visible liquid or oily droplets on the surface of the deodorant stick. As used herein, “visual detection” means that a human viewer can visually discern if the product had separated into two or more distinct layers or weeping with the unaided eye (except for standard corrective lenses adapted to compensate for near-sightedness, farsightedness, or astigmatism, or other corrected vision) in lighting at least equal to the illumination of a standard 100-watt incandescent white light bulb at 30 cm.

In Table 2, below, Example 1, which does not contain any polar organic solvents, showed a relatively high irritation score when compared to Examples 2 and 3. Both Examples 2 and 3 contained 63% polar organic solvents and could be less irritating with only 9% dropped sites, lower irritation scores, and more sites that had zero irritation, as compared to Example 1. However, Example 2, which included 5% sodium stearate was too hard and too little product was dispensed during the consumption test for it to be consumer preferred. In Example 3, the level of sodium stearate was decreased to 3.5% and had consumer acceptable hardness, consumption, and stability.

Examples 2 and 3 may be less consumer preferred than other examples disclosed herein because they have less thermal stability. Thermal stability can be performed under accelerated stability conditions of 50° C./75% relative humidity for about 12 hours. If a stick composition is not thermally stable, at least a portion of the stick composition will appear molten by visual detection. Thermal stability can be important because deodorant products can be exposed to relatively high temperatures during shipping, handling, or use. For instance, these temperature spikes can occur during transportation on a truck, storage in a non-climate-controlled warehouse, or when a user keeps the product in their gym bag inside a hot car on a summer day.

TABLE 2

| | Ex. 1 | Ex. 2 | Ex. 3 |
|---|---|---|---|
| Dipropylene glycol | 40-50 | 16.5 | 16.5 |
| Propylene glycol | 15-25 | 7 | 7 |
| 1,3-propanediol | | | |
| Glycerin | | 40 | 40 |
| Water, deionized | QS | QS | QS |
| Sodium stearate ^{a} | 5 | 5 | 3.5 |
| Perfume A | 2-4 | 4 | 4 |
| Miscellaneous minors ^{b} | 2.1 | 2.1 | 2.1 |
| Poloxamine 1307 | 3 | 3 | 3 |
| % polar organic solvents | 0 | 63.0 | 63.0 |
| (calc.) | | | |
| Skin Patch Study Results | | | |
| % dropped sites | 66 | 9 | 9 |
| Irritation score | 5.7 | 1.7 | 1.7 |
| % sites scored as zero | 8 | 51 | 51 |
| irritation | | | |
| Property Tests | | | |
| Hardness (Penetration mm*10) | | | |
| Consumption test (mg/cm²) | Pass | Fail | Pass |
| | | (low) | |
| Physical Stability Test | stable | stable | stable |

In Table 3, below, Examples 4 and 5 have 0% and 15.6%, respectively, polar organic solvents and the irritation scores are too high to be widely acceptable. Examples 6, 7, and 8 have lower irritation scores and % polar organic solvent that ranges from 46.1% to 61.7%.

TABLE 3

| | Ex. 4 | Ex. 5 | Ex. 6 | Ex. 7 | Ex. 8 |
|---|---|---|---|---|---|
| Dipropylene glycol | 45.5 | 38.4 | 25 | 18 | 17.8 |
| Propylene glycol | 18.6 | 15.7 | 10.1 | 7.15 | 7.07 |
| 1,3-propanediol | | | 22.5 | 30 | 30 |
| Glycerin | | 10 | 7.5 | 10 | 10 |
| Water, deionized | QS | QS | QS | QS | QS |
| Sodium stearate ^{a} | 5 | 5 | 4 | 4 | 4 |
| Perfume A | 3 | 3 | 3 | 3 | 3 |
| Miscellaneous minors ^{b} | 2.1 | 2.1 | 2.1 | 2.1 | 2.1 |
| Poloxamine 1307 | 3 | 3 | 3 | 3 | 3 |
| Antioxidant ^{c} | 0.03 | 0.03 | 0.03 | 0.03 | 0.28 |
| % polar organic solvents | 0 | 15.6 | 46.1 | 61.4 | 61.7 |
| (calc.) | | | | | |
| Skin Patch Study Results | | | | | |
| % dropped sites | 84 | 88 | 91 | 79 | 63 |
| Irritation score | 2.98 | 3.00 | 2.65 | 2.51 | 1.84 |
| % sites scored as zero | 5 | 5 | 7 | 16 | 26 |
| irritation | | | | | |

In Table 4, below, Ex. 9, 10, 12, and 13 were physically stable and are likely consumer acceptable. Ex. 11, which differs because it contains 3% sodium stearate instead of 4% and 3.5%, like Ex. 9 and 10, respectively. The 3% sodium stearate in combination with the solvents was not sufficient to stabilize the stick and weeping was observed.

TABLE 4

| | Ex. 9 | Ex. 10 | Ex. 11 | Ex. 12 | Ex. 13 |
|---|---|---|---|---|---|
| Dipropylene glycol | 17.8 | 17.8 | 17.8 | 32.3 | 31.5 |
| Propylene glycol | 7.07 | 7.07 | 7.07 | 9.94 | 9.71 |
| 1,3-propanediol | 30 | 30 | 30 | 18.75 | 18.75 |
| Glycerin | 10 | 10 | 10 | 6.25 | 6.25 |
| Water, deionized | QS | QS | QS | QS | QS |
| Sodium stearate ^{a} | 4 | 3.5 | 3 | 2.5 | 3.5 |
| Perfume A | 3 | 3 | 3 | 3.05 | 3.35 |
| Miscellaneous minors ^{b} | 2.1 | 2.1 | 2.1 | 0.9 | 0.9 |
| Poloxamine 1307 | 3 | 3 | 3 | 3 | 3 |
| Antioxidant ^{c} | 0.28 | 0.28 | 0.28 | 0.11 | 0.11 |
| % polar organic solvents | 61.7 | 61.7 | 61.7 | 37.2 | 37.8 |
| (calc.) | | | | | |
| Skin Patch Study Results | | | | | |
| % dropped sites | 63 | | | 63 | |
| Irritation score | 1.84 | | | 1.84 | |
| % sites scored as zero | 26 | | | 26 | |
| irritation | | | | | |
| Property Tests | | | | | |
| Physical Stability Test | stable | stable | unstable | stable | stable |
| | | | (weeping) | | |

Suppliers and/or Ingredient Details for Tables 2, 3, and 4:
a. OP-200V, from Hallstar® OP-200V is a vegetable-based sodium stearate, consisting primarily of the sodium salts of saturated C16 and C18 fatty acids, but also including a defined proportion of C20 and C22 fatty acids to increase the melt point.
b. Minors including PPG-3-myristyl ether, and tetrasodium EDTA
c. Octadecyl Di-t-butyl-4-hydroxyhydrocinnamate, commercially available as Tinogard® TS from BASF®

### Combinations:

A. A deodorant stick composition comprising:
a. from about 80% to about 95% of a solvent comprising:
   i. from about 20% to about 50%, preferably from about 22% to about 45%, and more preferably from about 25% to about 40%, by weight of the composition, of a polar organic solvent comprising 1,3 propanediol and/or glycerin;
   ii. a non-polar organic solvent comprising dipropylene glycol and/or propylene glycol;
   iii. water;
   wherein the ratio of polar organic solvent to non-polar organic solvent is from about 1:3 to about 3:1, preferably from about 1:2 to about 2:1, and more preferably from about 3:5 to about 1.7:1;
b. greater than 2% and less than 5%, preferably from about 2.25% to about 4.5%, and more preferably from about 2.5% to about 4% of a structurant; and
   wherein the composition comprises a hardness from about 80 to about 120 mm*10, preferably from about 85 to about 115 mm*10, and more preferably from about 85 to 100 mm*10.
B. The deodorant stick composition according to Paragraph A, wherein the polar organic solvent comprises 1,3 propanediol and glycerin.
C. The deodorant stick composition according to Paragraphs A-B, wherein the polar organic solvent consists of glycerin.
D. The deodorant stick composition according to Paragraphs A-C, wherein the composition comprises from about 3% to about 15%, preferably from about 4% to about 13%, and more preferably from about 5% to about 11% glycerin.
E. The deodorant stick composition according to Paragraphs A-D, wherein the composition comprises from about 25% to about 55%, preferably from about 30% to about 50%, more preferably from about 35% to about 45%, and even more preferably from about 38% to about 42% glycerin.
F. The deodorant stick composition according to Paragraphs A-E, wherein the structurant comprises sodium stearate.
G. The deodorant stick composition according to Paragraphs A-F, wherein the composition comprises from less than 50%, preferably less than 45%, more preferably less than 40%, and even more preferably less than 36%, by weight, of the non-polar organic solvent.
H. A deodorant stick composition comprising:
a. At least 80%, preferably at least 85%, even more preferably at least 90% of a solvent comprising water and organic solvent; wherein the organic solvent comprises:
   i. from about 25% to about 70%, preferably from about 30% to about 65%, more preferably from about 33% to about 63%, and even more preferably from about 35% to about 62%, by weight of the organic solvent of, a polar organic solvent comprising:
      1. from about 12% to about 40%, preferably from about 14% to about 40%, more preferably from about 15% to about 33%, and even more preferably from about 17% to about 30%, by weight of the composition, of 1,3 propanediol;
      2. from about 3% to about 15%, preferably from about 4% to about 13%, and even more preferably from about 5% to about 11%, by weight of the composition, of glycerin;
   ii. a non-polar organic solvent comprising:
      1. from about 5% to about 45%, preferably from about 10% to about 40%, more preferably from about 15% to about 35%, by weight of the composition, of a dipropylene glycol;
      2. from about 3% to about 15%, preferably from about 5% to about 13%, more preferably from about 6% to about 12%, and even more preferably from about 7% to about 10%, by weight of the composition, of a propylene glycol;
b. greater than 2% and less than 5%, preferably from about 2.25% to about 4.5%, and more preferably from about 2.5% to about 4% of a structurant comprising sodium stearate; and
   wherein the composition comprises a hardness from about 80 to about 120 mm*10, preferably from about 85 to about 115 mm*10, and more preferably from about 85 to 100 mm*10.
I. The deodorant stick composition according to Paragraphs A-H, wherein the polar organic solvent comprises a ratio of 1,3 propanediol to glycerin of from about 1:3 to about 5:1, preferably from about 1:2 to about 4:1, and more preferably from about 3:5 to about 3:1.
J. The deodorant stick composition according to according to Paragraphs A-I, wherein the composition further comprises an antimicrobial chosen from piroctone olamine, octenidine HCl, hexamidine, magnesium carbonate, zinc carbonate, thymol, magnesium hydroxide, dead sea salt, magnesium hydroxide and magnesium carbonate hydroxide, calcium carbonate, polyvinyl formate, salycilic acid, niacinamide, phenoxyethanol, eugenol, linolenic acid, dimethyl succinate, citral, triethyl citrate, sepiwhite, baking soda, partially carbonated magnesium hydroxide, magnesium carbonate hydroxide, cinnamon essential oil, cinnamon bark essential oil, cinnamic aldehyde, or mixtures thereof.
K. The deodorant stick composition according to Paragraph J, wherein the antimicrobial comprises octenidine HCl.
L. The deodorant stick composition according to Paragraphs A-K, wherein the composition is physically stable and thermally stable.
M. The deodorant stick composition according to Paragraphs A-L, wherein the deodorant stick composition has a consumption of about 3 to about 4.5 mg/cm², preferably about 3.5 to about 4 mg/cm², according to the Consumption Test Method.
N. Use of a deodorant composition according Paragraphs A-M. for reducing underarm irritation.
O. Use of a deodorant composition according Paragraphs A-M, for preventing underarm irritation.
P. Use of a deodorant composition according to according to Paragraphs A-M, for providing wetness protection.
Q. Use of a deodorant composition comprising the solvent according to Paragraphs A-M, for providing wetness protection.
R. Use of a deodorant composition according to according to Paragraphs A-M, for providing underarm malodor control.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A deodorant stick composition comprising:
a. from about 80% to about 95% of a solvent comprising:
i. from about 20% to about 50%, by weight of the composition, of a polar organic solvent chosen from 1,3 propanediol, glycerin, or mixtures thereof;
ii. a non-polar organic solvent comprising dipropylene glycol and/or propylene glycol;
iii. water;
wherein the ratio of polar organic solvent to non-polar organic solvent is from about 1:3 to about 3:1;
b. greater than 2% and less than 6% of a structurant; and
wherein the composition comprises a hardness from about 80 to about 120 mm*10.

2. The deodorant stick composition of claim 1, wherein the polar organic solvent comprises 1,3 propanediol and glycerin.

3. The deodorant stick composition of claim 2, wherein the polar organic solvent comprises a ratio of 1,3 propanediol to glycerin of from about 1:2 to about 2:1.

4. The deodorant stick composition of claim 2, wherein the polar organic solvent comprises a ratio of 1,3 propanediol to glycerin of from about 3:5 to about 1:1.

5. The deodorant stick composition of claim 2, wherein the composition comprises from about 3% to about 15% glycerin.

6. The deodorant stick composition of claim 1, wherein the structurant comprises sodium stearate.

7. The deodorant stick composition of claim 1, wherein the composition comprises from less than 50% of the non-polar organic solvent.

8. The deodorant stick composition according to claim 1, wherein the composition further comprises an antimicrobial chosen from piroctone olamine, octenidine HCl, hexamidine, magnesium carbonate, zinc carbonate, thymol, magnesium hydroxide, dead sea salt, magnesium hydroxide and magnesium carbonate hydroxide, calcium carbonate, polyvinyl formate, salycilic acid, niacinamide, phenoxyethanol, eugenol, linolenic acid, dimethyl succinate, citral, triethyl citrate, sepiwhite, baking soda, partially carbonated magnesium hydroxide, magnesium carbonate hydroxide, cinnamon essential oil, cinnamon bark essential oil, cinnamic aldehyde, or mixtures thereof.

9. The deodorant stick composition according to claim 8, wherein the antimicrobial comprises octenidine HCl.

10. The deodorant stick composition of claim 1, wherein the composition is physically stable and thermally stable.

11. A deodorant stick composition comprising:
a. at least 85% of a solvent comprising water and organic solvent; wherein the organic solvent comprises:
i. from about 25% to about 70%, by weight of the organic solvent of, a polar organic solvent comprising:
1. from about 12% to about 40% of a 1,3 propanediol;
2. from about 3% to about 15% of a glycerin;
3. optionally one or more additional organic solvents;
ii. a non-polar organic solvent comprising:
1. from about 5% to about 45% of a dipropylene glycol;
2. from about 3% to about 15% of a propylene glycol;
b. greater than 2% and less than 5% of a structurant comprising sodium stearate; and
wherein the composition comprises a hardness from comprises a hardness from about 80 to about 120 mm*10.

12. The deodorant stick composition of claim 11, wherein the composition is physically stable and thermally stable.

13. The deodorant stick composition of claim 11, wherein the deodorant stick composition has a consumption of about 3 to about 4.5 mg/cm².

14. The deodorant stick composition of claim 11, wherein the deodorant stick comprises a hardness of from about 85 to about 115 mm*10.

15. The deodorant stick of claim 11, wherein the deodorant stick comprises from about 10% to about 30% water.

16. The deodorant stick of claim 11, wherein the deodorant stick comprises a water activity of from about 0.4 to about 0.85.

17. The deodorant stick composition according to claim 11, wherein the composition further comprises an antimicrobial chosen from piroctone olamine, octenidine HCl, hexamidine, magnesium carbonate, zinc carbonate, thymol, magnesium hydroxide, dead sea salt, magnesium hydroxide and magnesium carbonate hydroxide, calcium carbonate, polyvinyl formate, salycilic acid, niacinamide, phenoxyethanol, eugenol, linolenic acid, dimethyl succinate, citral, triethyl citrate, sepiwhite, baking soda, partially carbonated magnesium hydroxide, magnesium carbonate hydroxide, cinnamon essential oil, cinnamon bark essential oil, cinnamic aldehyde, or mixtures thereof.

18. The deodorant stick composition according to claim 17, wherein the antimicrobial comprises octenidine HCl.

19. The deodorant stick composition of claim 11, wherein the composition is physically stable and thermally stable.
